(19) United States
(12) Patent Application Publication
(10) Pub. No.: US 2010/0126507 A1
(43) Pub. Date: May 27, 2010

(54) MULTI-DOSE INHALER DEVICE

(75) Inventors: Amar Lulla, Maharashtra (IN); Geena Malhotra, Maharashtra (IN); Xerxes Rao, Maharashtra (IN)

Correspondence Address: DYKEMA GOSSETT PLLC, FRANKLIN SQUARE, THIRD FLOOR WEST, 1300 I STREET, NW, WASHINGTON, DC, 20005, US

(73) Assignee: CIPLA LIMITED, Mumbai, Mumbai Central (IN)

(21) Appl. No.: 12/450,307

(22) PCT Filed: Mar. 20, 2008
(86) PCT No.: PCT/GB2008/001014

§ 371 Date: Jan. 22, 2010

(30) Foreign Application Priority Data
Mar. 21, 2007 (IN) 0520/MUM/2007
Sep. 7, 2007 (IN) 1718/MUM/2007

(51) Int. Cl. A61M 15/00

(52) U.S. Cl. 128/203.15

(57) ABSTRACT

The present invention provides an inhaler device (1) for dispensing multiple doses of medicament, the inhaler comprising medicament extraction facilitating means (22) for locating adjacent or in a compartment (42) of a received medicament cartridge and thereby allowing an extraction of medicament from the compartment; and operating means (54) moveable in first and second directions by a user. The medicament extraction facilitating means is movable from a retracted position to an extraction position in response to movement of the operating means in the first direction. The extraction position allows extraction of medicament from said cartridge compartment upon inhalation by a user, and the retracted position allows advancement of the medicament cartridge. The medicament cartridge is driven by the operating means so as to advance a cartridge compartment into a predetermined position relative to the extraction facilitating means in response to movement of the operating means in the second direction.

The present invention relates to inhalers for the administering of medicament. The invention relates particularly to multi-dose inhalers and more particularly to inhalers which are used with piercable sealed cartridges having a plurality of medicament compartments. Such inhalers allow for the administration of predetermined doses of medicament (typically a dry powder medicament).

A wide variety of multi-dose inhalers are presently available on the market and they are designed for the inhalation of predetermined multiple doses of medicament by a patient. A problem often associated with multi-dose inhalers relates to the need for a cartridge comprising multiple doses of medicament to be advanced and a sealed cartridge compartment to be pierced before a dose of medicament can be dispensed. This requirement has in the past resulted in complicated and expensive mechanisms for allowing a multiple dose cartridge to be conveniently used by a patient.

It is an object of the present invention to provide an inhaler device which is less expensive and less complicated in construction than prior art multi-dose inhaler devices.

A first aspect of the present invention provides an inhaler device for dispensing medicament, the inhaler device comprising medicament extraction facilitating means comprising a resiliently displaceable member.

A second aspect of the present invention provides an inhaler device for dispensing medicament, the inhaler device comprising medicament extraction facilitating means wherein said means is sprung biased or is operated by a cam mechanism and displaced over a pivot. Ideally, said means is biased into a medicament extraction position allowing extraction of medicament from a medicament cartridge. Preferably said means is sprung biased by virtue of the resiliently deflectable character of the material from which said means is manufactured.

A third aspect of the present invention provides an inhaler device for dispensing multiple doses of medicament, the inhaler comprising means for receiving a medicament cartridge comprising a plurality of compartments containing medicament; medicament extraction facilitating means for locating adjacent or in a compartment of a received cartridge and thereby allowing an extraction of medicament from the compartment; and operating means moveable in first and second directions by a user; wherein the medicament extraction facilitating means is movable from a retracted position to an extraction position in response to movement of the operating means in the first direction, the extraction position of the medicament extraction facilitating means allowing extraction of medicament from said cartridge compartment upon inhalation by a user, and the retracted position allowing advancement of the medicament cartridge, and wherein the medicament cartridge is driven by the operating means so as to advance a cartridge compartment into a predetermined position relative to the medicament extraction facilitating means in response to movement of the operating means in the second direction.

Preferably, the medicament extraction facilitating means also moves from the extraction position to the retracted position in response to movement of the operating means in the second direction.

The medicament extraction facilitating means may preferably be formed from delrin, but ABS, nylon, polycarbonate, HDPE, LDPE or acetal may also be used.

It will be understood therefore that an inhaler device according to the present invention allows for a comparatively simple operation by a user through appropriate movement of the operating means. If a user wishes to inhale a dose of medicament from a cartridge within the inhaler device, then the user need only move the operating means in a first direction so as to move the extraction facilitating means to an extraction position in which medicament may be extracted from the cartridge compartment. A user may then readily move the operating means in a second direction so as to advance or index the next dose of medicament to an appropriate position. Typically, this position will be one adjacent the medicament extraction facilitating means. It will be appreciated that the operating means may drive both the advancement of the cartridge and movement of the medicament extraction facilitating means.

Alternatively, rather than the operating means driving the medicament extraction facilitating means into an extraction position, the medicament extraction facilitating means may be driven by the operating means so as to move the medicament extraction facilitating means from the extraction position. The medicament extraction facilitating means may be moved to a retracted position which allows for the advancement of the medicament cartridge.

Whilst the operating means drives the medicament extraction facilitating means to one of the extraction and retracted positions, biasing means may be provided for moving the medicament extraction facilitating means to the other of the extraction and retracted positions. The biasing means may comprise a resilient elastic member. The resilient elastic member may be provided as a cantilever arm, with one end thereof fixed to a housing of the inhaler device and a free end thereof being provided with the medicament extraction facilitating means.

The operating means may, alternatively, drive the medicament extraction facilitating means to both the extraction and retracted positions. Biasing means may also be provided to bias the medicament extraction facilitating means towards one of the extraction and retracted positions.

The operating means may drive the medicament extraction facilitating means through use of a cam arrangement. Ideally, a cam is provided on the operating means. The cam may be provided as a ramp surface. The arrangement may be such that, when the operating member is moved in the second direction, the ramp surface moves laterally in abutment with the medicament extraction facilitating means and thereby displaces the medicament extraction facilitating means. Preferably, the medicament extraction facilitating means is provided with a roller or bearing before abutment with the cam.

The medicament extraction facilitating means may be provided with a tubular element for locating in a cartridge compartment when the medicament extraction facilitating means is located in the extraction position. A free end of the tubular element may be provided with a piercing portion. The piercing portion may be of a frusto-conical shape. Furthermore, the arrangement of the medicament extraction facilitating means may be such that, when the medicament extraction facilitating means is located in the extraction position, the tubular element is in fluid communication with a mouthpiece of the inhaler device. This fluid communication may be made through an aperture or discontinuity in the operating means. This aperture or discontinuity is ideally aligned with a flow path through the mouthpiece when the operating means has been moved in the first direction so as to drive the medicament extraction facilitating means into the extraction position.

The mouthpiece may also include an opening (through which medicament is inhaled) which is provided with three helical guide vanes for imparting a swirl motion onto medicament/fluid to be inhaled. Other means for imparting a swirl motion on medicament/fluid may be provided.

It is also preferable for the operating means to close a flow path through the mouthpiece when the operating means is moved in the second direction. Ideally, the aperture or discontinuity is moved out of alignment with the mouthpiece flow path when the operating means is moved in the second direction. Fluid communication between the mouthpiece and the extraction facilitating means is ideally prevented when the medicament extraction facilitating means is in the retracted position.

Furthermore, it is preferable for the medicament extraction facilitating means to comprise a venturi.

It is also desirable for ratchet means to be provided for allowing the operating means to drive the medicament cartridge. The operating means may be provided with a ratchet tooth so that the medicament cartridge is moved in response to movement of the operating means in the second direction, but is not moved in response to movement of the operating means in the first direction. Said ratchet tooth may be arranged so as to engage in turn with each of a plurality of ratchet teeth provided on the medicament cartridge.

Ideally, said first direction is opposite to said second direction. It is also preferable for movement of the operating means in said first and second directions to comprise a movement of the operating means along a part-circular path. The part-circular path is ideally concentrically positioned relative to an annular arrangement of medicament compartments of the medicament cartridge.

An element of the operating means may extend through a slot in a housing of the inhaler device. The slot is ideally configured so as to allow movement of said element between opposite ends of the slot and thereby move the operating means in said first and second directions. Retaining means may be provided for preventing movement of said element of the operating means. Said retaining means may be provided on a cover for the mouthpiece, possibly as part of a cover for the mouthpiece.

The inhaler device may be provided with means for retaining a medicament cartridge in a required position. The retaining means may comprise the roller, wheel or bearing to press against the cartridge whilst allowing movement of the cartridge. The retaining means may be sprung biased so as to press against the medicament cartridge. The retaining means is preferably made from delrin, but may be made from ABS, nylon, polycarbonate, HDPE, LDPE or acetal. The inhaler may also comprise means for driving a dose counter in response to movement of a medicament cartridge. Said drive means may comprise one or more teeth or detents for engaging a medicament cartridge. The drive means may also comprise a gear wheel for engaging means for displaying a dose count.

Embodiments of the present invention will now be described with reference to the accompanying drawings, in which:

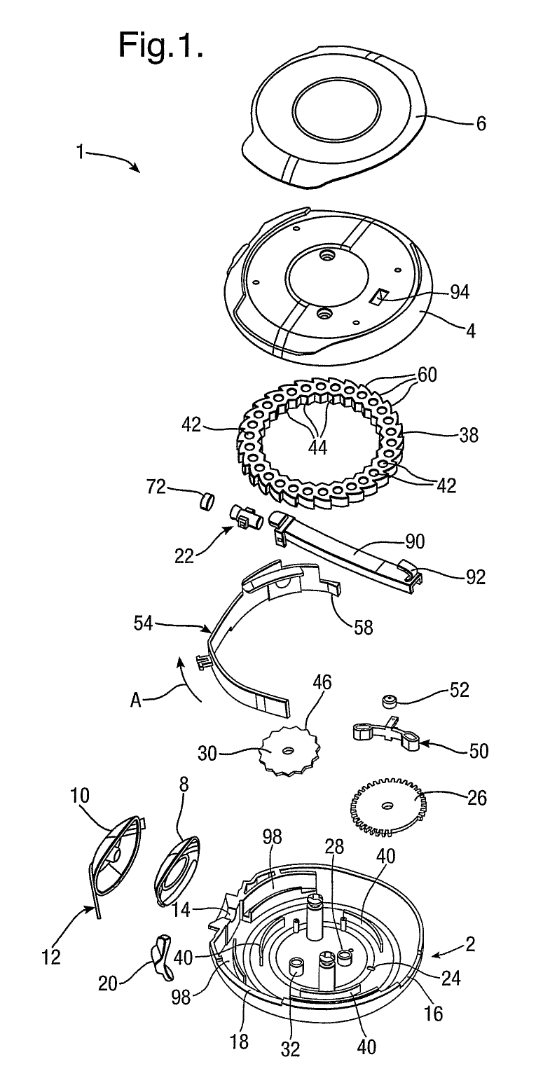

FIG. 1 is an exploded view of a first inhaler device according to the present invention;

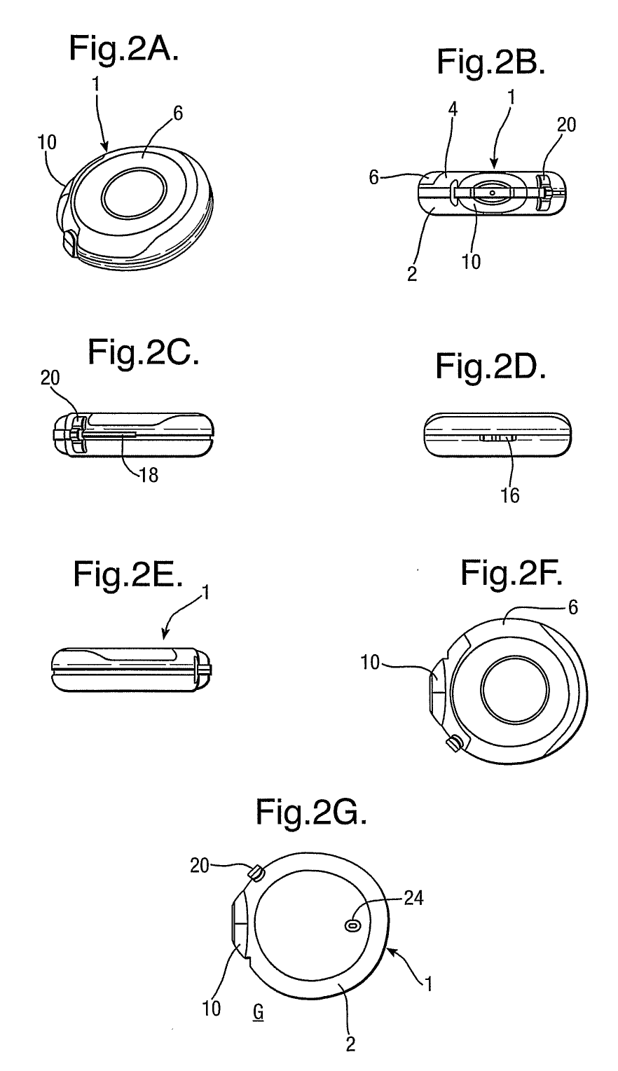

FIGS. 2A-G are perspective, front, right side, rear, left side, top and bottom views respectively of the assembled inhaler device shown in FIG. 1;

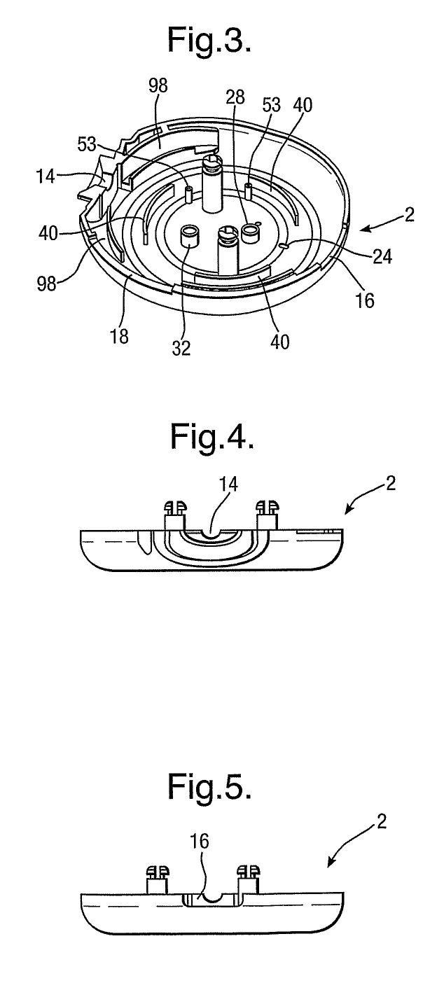

FIG. 3 is a perspective view of a base of the inhaler shown in FIG. 1;

FIG. 4 is a front view of the base shown in FIG. 3;

FIG. 5 is a rear view of the base shown in FIG. 3;

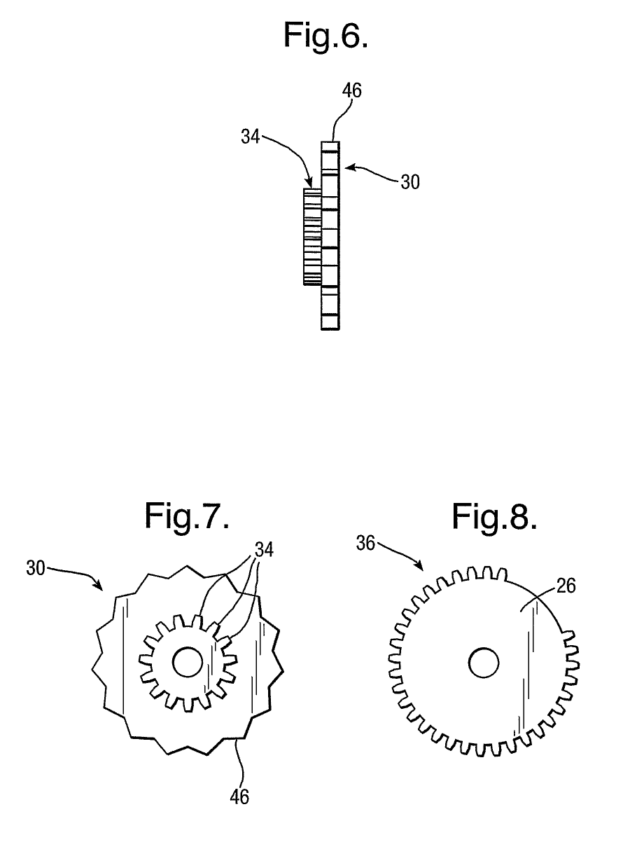

FIG. 6 is a side view of a dose counter drive gear;

FIG. 7 is a bottom view of the drive gear of FIG. 6;

FIG. 8 is a bottom view of a dose indicator wheel;

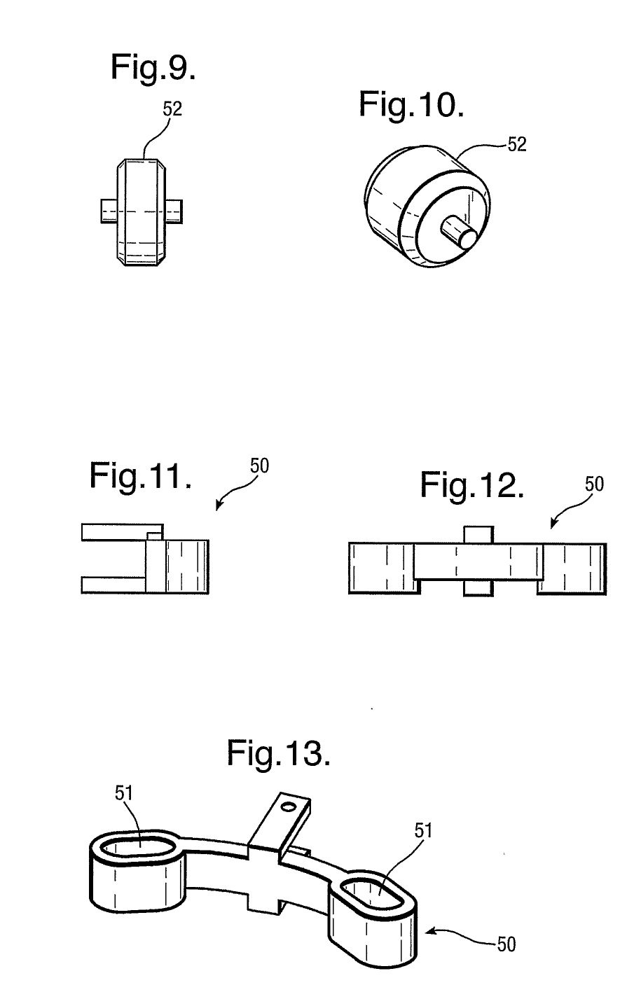

FIG. 9 is an end view of a roller for a cartridge centring mechanism;

FIG. 10 is a perspective view of the roller of FIG. 9;

FIG. 11 is a side view of a cartridge centring mechanism (shown without the roller of FIG. 9);

FIG. 12 is a rear view of the centring mechanism of FIG. 11;

FIG. 13 is a perspective view of the centring mechanism of FIG. 11;

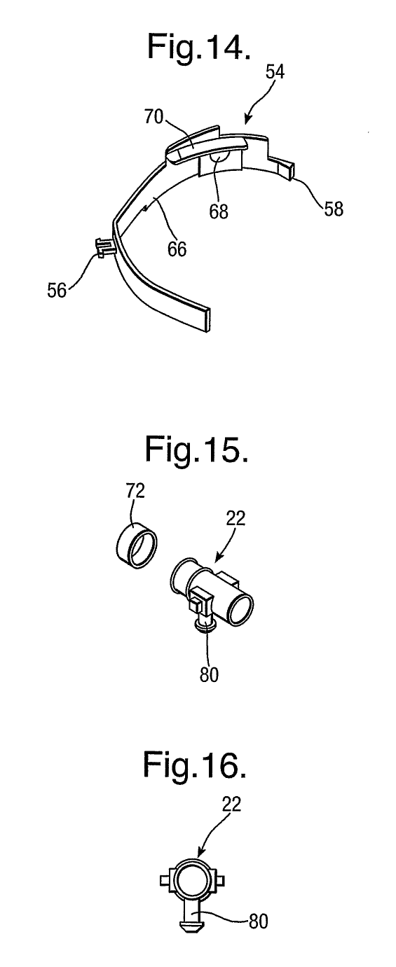

FIG. 14 is a perspective view of inhaler operating means comprising a cam strip;

FIG. 15 is a perspective view of a spiking component and associated roller;

FIG. 16 is an end view of the spiking component of FIG. 15;

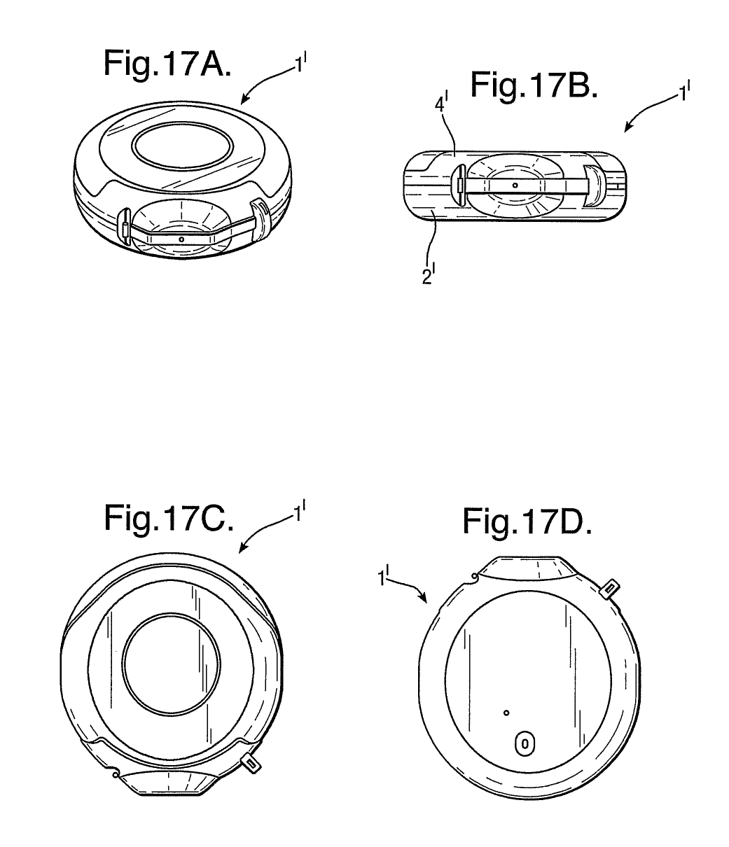

FIGS. 17A-D are persepective, front, top and bottom views respectively of an assembled second inhaler device according to the present invention;

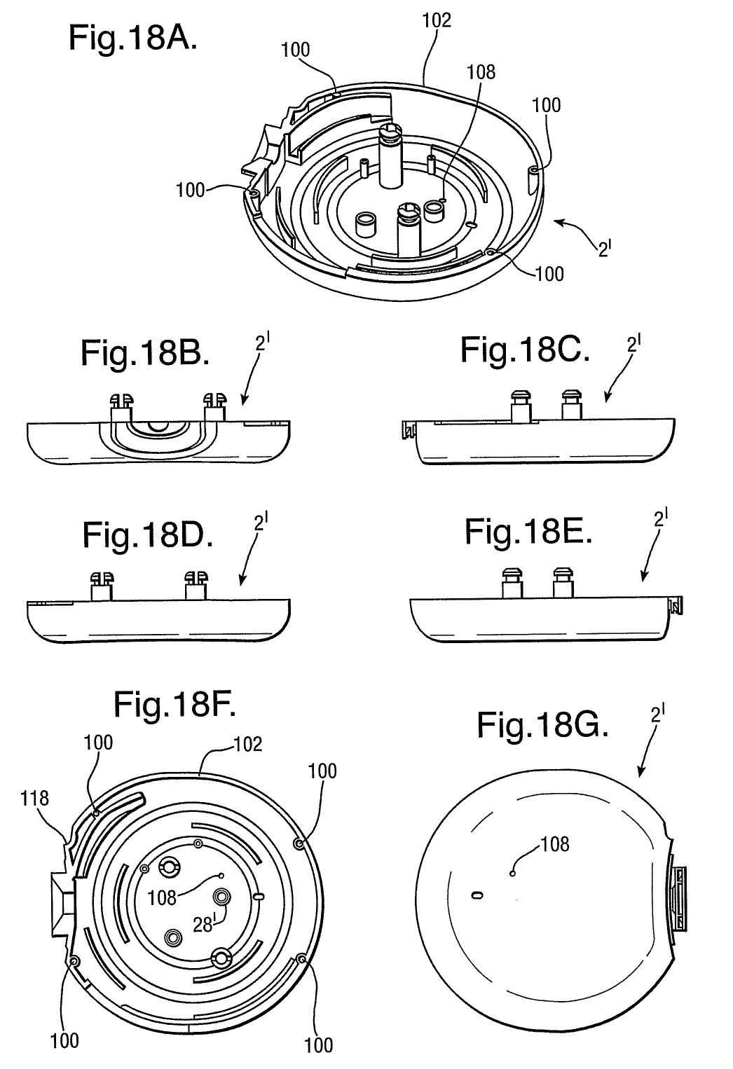

FIGS. 18A-G are perspective, front, right side, rear, left side, top and bottom views respectively of the base of the second inhaler device;

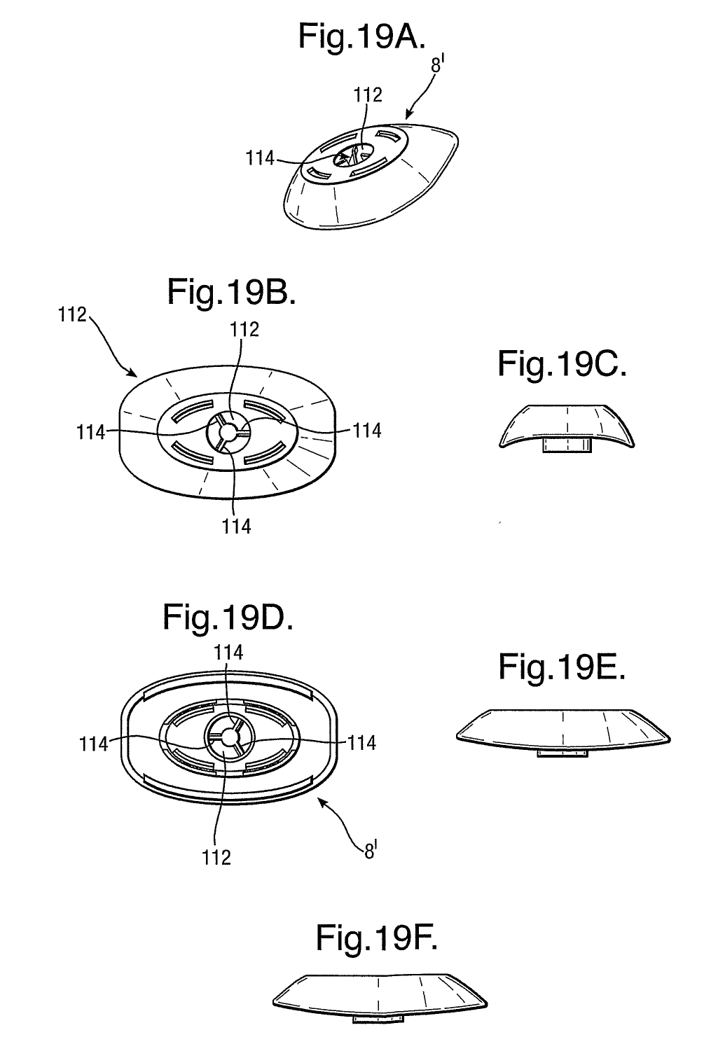

FIGS. 19 A-F are perspective, front, left side, rear, top and bottom views respectively of a mouthpiece of the second inhaler device;

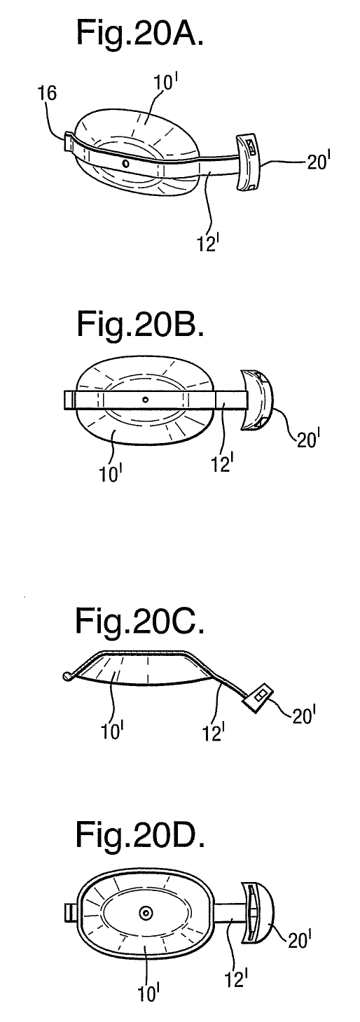

FIGS. 20A-D are perspective, front, bottom and rear views respectively of a mouthpiece cover of the second inhaler device;

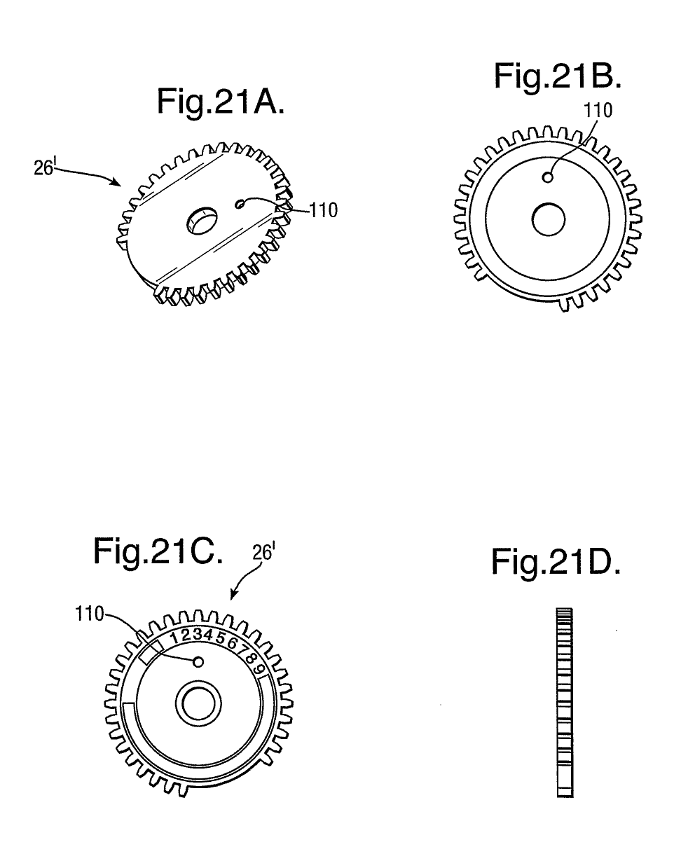

FIGS. 21A-D are perspective, top, bottom and side views respectively of a dose counter wheel 26 of the second inhaler device;

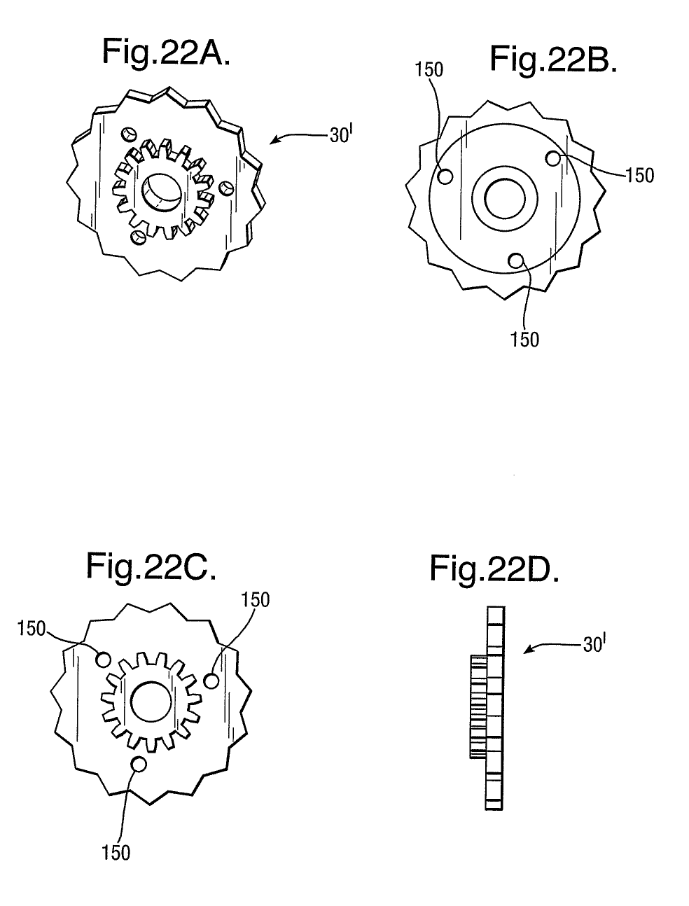

FIGS. 22A-D are perspective, top, bottom and side views respectively of a drive gear of the second inhaler device;

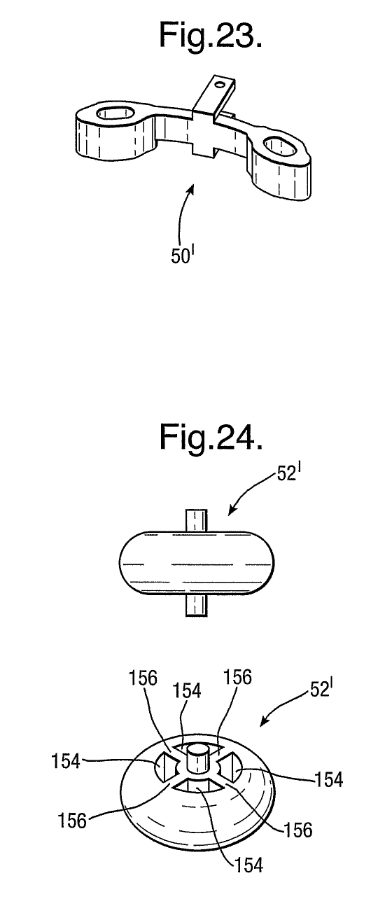

FIG. 23 is a perspective view of a medicament cartridge centring mechanism and associated roller of the second inhaler device;

FIG. 24 is a side view of the roller shown in FIG. 23;

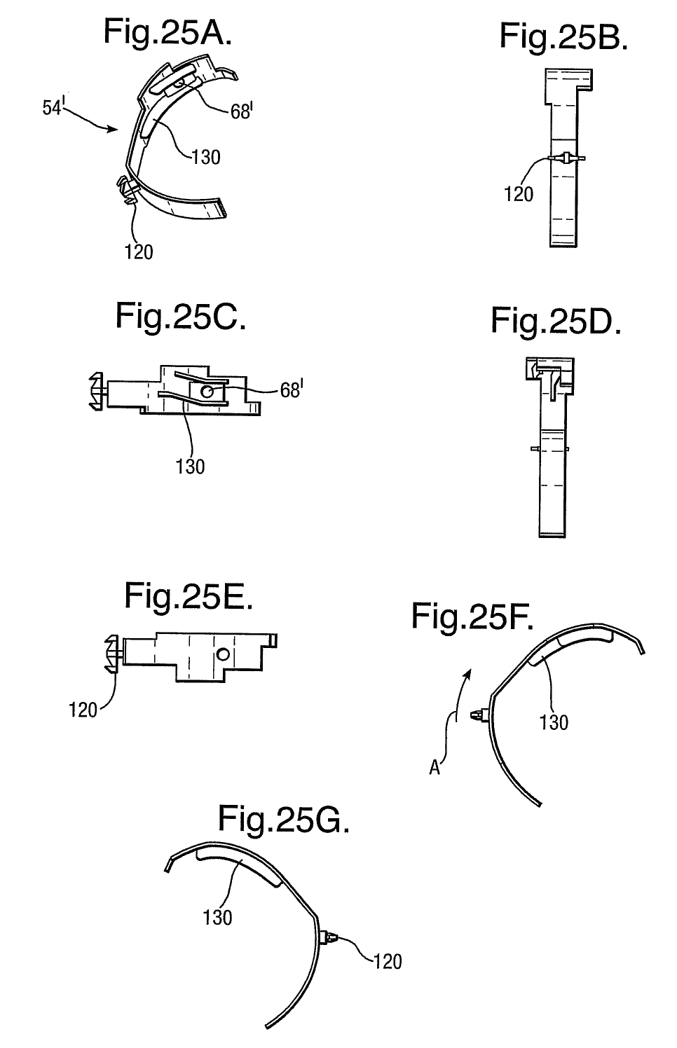

FIGS. 25A-G are perspective, front, right side, rear, left side, top and bottom views respectively of an operating member of the second inhaler device;

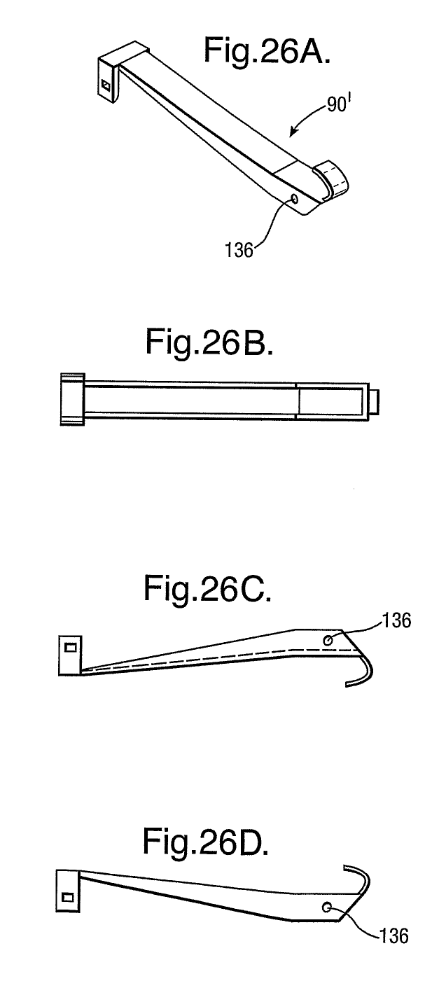

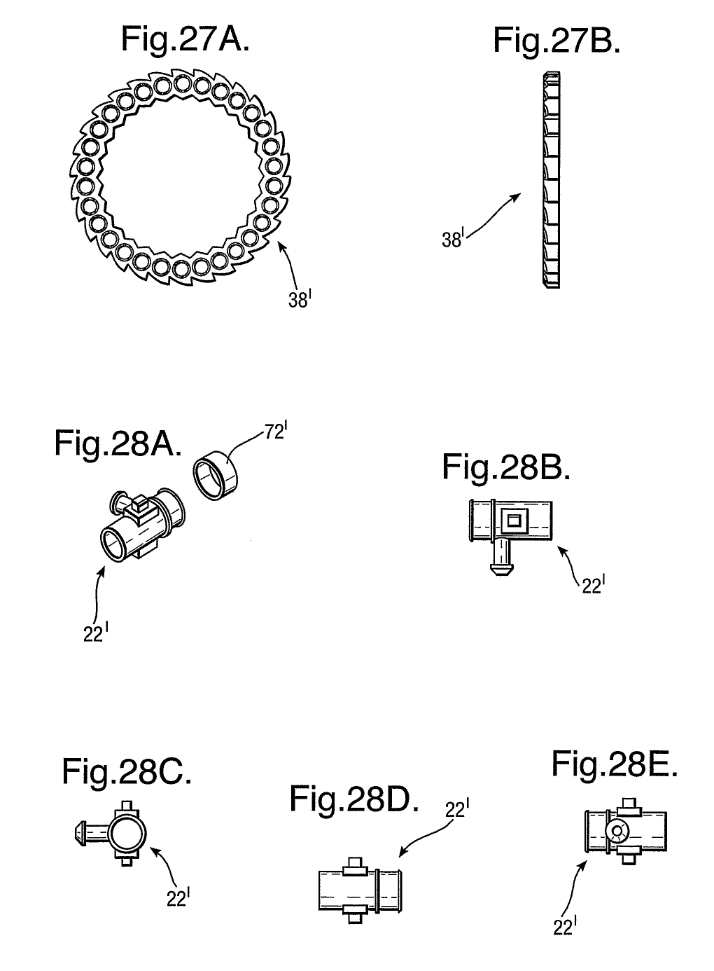

FIGS. 26A-D are perspective, bottom, right side and left side views respectively of a support arm of the second inhaler device;

FIGS. 27A & B are top and side views respectively of a medicament cartridge of the second inhaler device;

FIGS. 28A-E are, respectively, a perspective view of a spiking component of the second inhaler device and a roller to be mounted to said component, and right side, rear, top and bottom views of the spiking component alone;

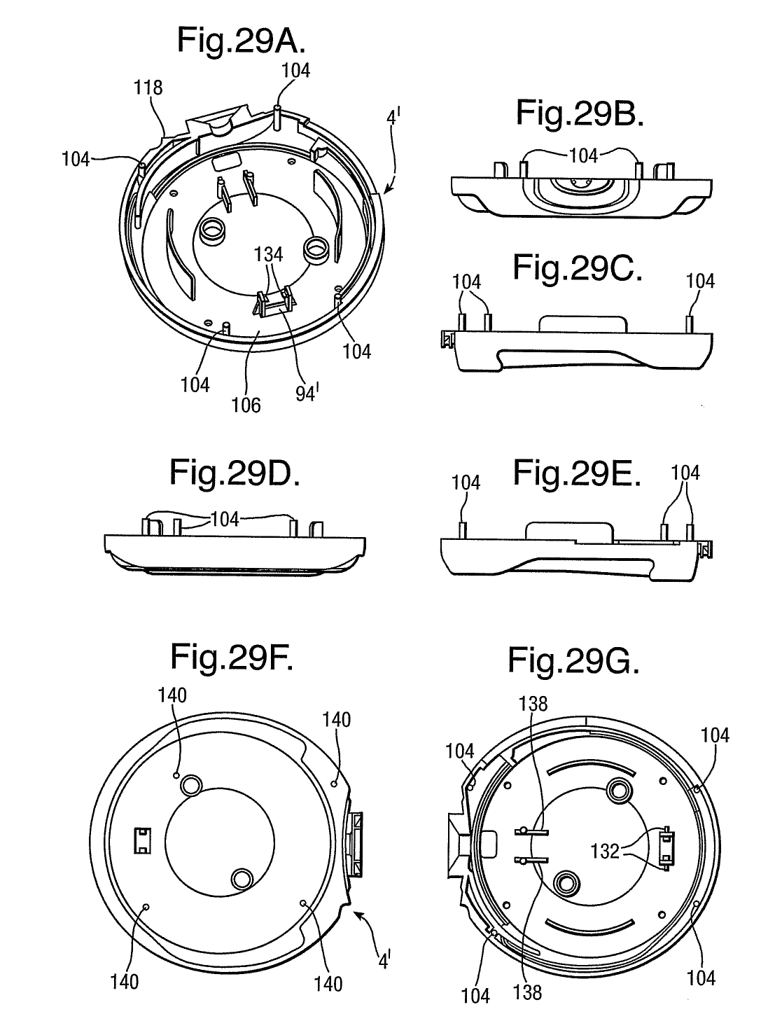

FIGS. 29A-G are perspective, front, right side, rear, left side, top and bottom views respectively of a middle cover of the second inhaler device; and

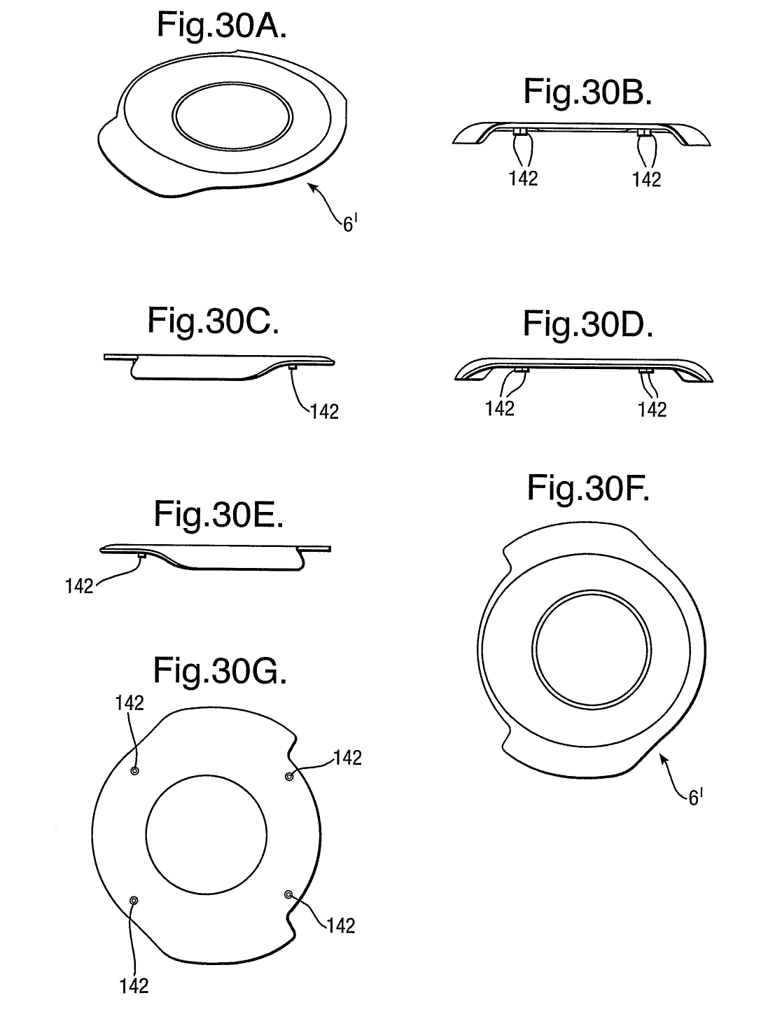

FIGS. 30A-G are perspective, front, right side, rear, left side, top and bottom views respectively of a top cover of the second inhaler device.

An exploded view of a first inhaler device 1 is shown in FIG. 1 of the accompanying drawings. Various views of the inhaler device 1 in an assembled condition are shown in FIGS. 2A-G. Individual components of the first inhaler device 1 are also shown in FIGS. 3 to 16.

The inhaler device 1 comprises an outer housing and a plurality of internal components including a cartridge having a plurality of compartments containing medicament. The housing comprises a base 2 (also shown in FIGS. 3 to 5), a middle cover 4 and a top cover 6. The housing is provided with a mouthpiece 8 and a mouthpiece Cover 10. An aperture 14 in the front side of the housing allows for fluid communication through the mouthpiece 8 between the interior and exterior of the inhaler housing. In use, a user of the inhaler device 1 places the mouthpiece to the lips of their mouth and inhales through the mouthpiece so as to extract and inhale medicament. In an alternative embodiment, the mouthpiece 8 may be replaced with a construction suitable for nasal inhalation.

A further aperture 16 is provided in the rear of the housing which allows for a flow of air into the housing during inhalation of medicament by a user. The housing is also provided with a slot 18 along which a knob 20 may be moved so as to advance a cartridge of medicament within the housing and so as to displace a spiking component 22. The mouthpiece cover 10 is pivotally mounted to the knob by means of a hinge arrangement 12. Accordingly, the cover 10 and the knob 20 move together during actuation of the device. A single hand can be used to open the mouthpiece cover 10 and actuate the device by moving the knob 20. Alternatively, the mouthpiece cover 10 may be pivotally mounted to the housing by means of a hinge arrangement 12. An aperture or window 24 (see also FIG. 2G) is also provided in the bottom of the base 2 so that an indication of the number of doses used or the number of doses remaining can be seen provided on a dose counter wheel 26 located within the housing.

The wheel 26 is located on and rotatably attached to a first boss 28 upstanding from an interior surface of the base 2. A drive gear 30 for the counter wheel 26 is rotatably mounted to a second boss 32. The second boss upstands from the same surface as the first boss 28 and is spaced from the first boss 28 so that teeth 34 (see FIGS. 6 and 7) on the lower side of the drive gear 30 engage with teeth 36 on the circumferential edge of the outer wheel 26. In use, the drive gear 30 rotates with a medicament cartridge and, due to the engagement of the teeth 34, 36, rotates the counter wheel 26 so that an appropriate indicator provided on the underside of the wheel 26 may be viewed through the window 24 and thereby provide a user with an indication of the remaining doses or the doses used.

A medicament cartridge 38 locates on three equi-spaced support members 40 provided on the base 2. The medicament cartridge 38 locates above the counter wheel 26 but in the same plane as the drive gear 30.

The cartridge 38 is provided as an annular ring having a plurality of compartments 42. Each compartment contains medicament in a powder form. The annular ring comprises two operative walls—an outer operative wall and an inner operative wall. Both walls have a plurality of teeth integral therewith. The inner wall comprises a plurality of integral teeth 44 which engage with a plurality of teeth 46 provided on the perimeter of the drive gear 30. The arrangement is such that a small number of teeth 46 are engaged at any one time with the same number of teeth 44 of the medicament cartridge 38. Thus, as the cartridge 38 rotates whilst supported on the support members 40, the drive gear 30 is rotated on the boss 32 by virtue of the engagement between the aforesaid teeth 44, 46.

The engagement of the drive gear 30 with the medicament cartridge 38 also assists in retaining the cartridge 38 in a required position within the inhaler housing. The position of the medicament cartridge 38 is further retained by means of a centring mechanism 50 which presses a roller 52 against the teeth 44. Apertures 51 in the centring mechanism 50 are snap-fitted to posts 53 upstanding from the base 2. The posts 53 have a flange (not shown) provided on their free ends so as to retain the centring mechanism 50 on the base 2.

The centring mechanism 50 is made from a resiliently displaceable member so as to allow the roller 52 to be biased against the cartridge 38. In this way, undesirable lateral movement and/or rotation of the medicament cartridge 38 is prevented.

The medicament cartridge 38 is indexed from one medicament compartment 42 to the next through movement by the inhaler user of an operating member 54 (an enlarged view of which is provided in FIG. 14). An element 56 of the operating member 54 extends through the slot 18 and attaches with the knob 20. The operating member 54 comprises a ratchet tooth 58 which engages with a plurality of ratchet teeth 60 provided on the outer operative wall of the medicament cartridge 38. The configuration and engagement of the ratchet teeth 58, 60 is such that, when the operating member 54 is moved in the direction of arrow A (see FIG. 1) the ratchet tooth 58 of the operating member 54 abuts against an adjacent ratchet tooth 60 of the cartridge 38 and rotates the cartridge 38′ in the direction of arrow A. The extent of movement of the operating member 54 allowed by the slot 18 indexes the cartridge 38 by one compartment 42 (i.e. to the next compartment 42). The movement of the cartridge 38 will also be understood to drive the dose counter wheel 26 as already described above.

When the operating member 54 is moved in an opposite direction to that indicated by arrow A, the configuration of the ratchet mechanism allows the ratchet tooth 58 on the operating member 54 to ride over the ratchet teeth 60 of the cartridge 38. The cartridge 38 is therefore not rotated by movement of the operating member 54 in a direction opposite to that indicated by arrow A.

The operating member 54 further comprises a sealing face plate 66 which aligns with the flow path 14 associated with the mouthpiece 8 when the operating member 54 has been moved to the fullest extent in the direction indicated by arrow A. Furthermore, the operating member 54 is provided with an aperture 68 which aligns with the flow path 14 when the operating member 54 has been moved to the fullest extend in the direction opposite to that indicated by arrow A.

The operating member 54 yet further comprises a camming member/strip or ramp 70 which engages with a roller 72 mounted on the spiking component 22. In the assembled inhaler 1 the camming strip 70 is located above the roller 72 and the arrangement is such that, when the operating member 54 is moved in a direction opposite to that indicated by arrow A, the roller 72 rolls against the underside of the cam strip 70 and is pressed downwardly by the camming strip 70 so as to locate the spiking component within a previously advanced compartment 42 of medicament. In so locating the spiking component 22, a seal provided across the top of the compartment 42 is perforated by the spiking component 22. The spiking component 22 comprises a tubular element 80 which is located in fluid communication with the aperture 68 and flow path 14. Thus, with the operating member 54 having been moved in a direction opposite to that indicated by arrow A, the tubular element 80 is located in a compartment containing medicament and the aperture 68 of the operating member 54 is aligned with the mouthpiece flow path 14. Medicament may then be inhaled from the medicament compartment from the mouthpiece.

When the operating member 54 is moved back in the direction indicated by arrow A, the aperture 68 is moved out of alignment with the mouthpiece flow path 14. The flow path 14 is then covered by the sealing plate 66 of the operating member 54. Also, as the operating member 54 is moved in the direction of arrow A, the downward pressure applied by the cam 70 is released and the spiking component 22 is moved upwardly by the cam movement/spring bias of a support arm 90 upon an end of which the spiking component 22 is mounted. At an opposite end of the arm 90, a hooking element 92 is provided for securing the arm 90 to the middle cover 4 through an aperture 94 provided in said cover 4. The arm 90 is therefore provided as a cantilever. The arm 90 is a resiliently displaceable member, and is preferably made from delrin, but may be made from nylon, polycarbonate or acetal.

The above materials, among others, are also suitable for the manufacture of various other parts of the device, including, but not limited to; the spiking component 22, the hooking element 92, the cartridge centring mechanism 50 and the rollers 52, 72.

It will be understood that sliding the operating member 54 in the direction of arrow A in FIG. 1 results in moving the inhaler 1 into a closed position, whilst sliding the operating member 54 in the direction opposite to that indicated by arrow A results in moving the inhaler 1 into an open position.

The operating member 54 is located within the inhaler housing in a groove 98. The groove 98 allows the operating member 54 to be displaced along the inner side of the housing (i.e. circumferential movement) whilst limiting undesirable radial movement. Although not illustrated in the drawings, the spiking component 22 comprises a venturi which is positioned so that inhalation by a user causes medicament to travel up the tubular element 80 and into the venturi before eventually entering the lungs of the user.

The inhaler 1 may be sealed so that once the medicament within the cartridge 38 has been used, the inhaler 1 must be discarded. Alternatively, the inhaler 1 may be provided with a lid which allows access to the interior of the inhaler and thereby allows for a replacement of the used cartridge with a fresh cartridge. Accordingly, it will be understood that the inhaler 1 may incorporate a replaceable medicament cartridge so that, once the cartridge is exhausted, it can be replaced with a medicament cartridge which is fully loaded with medicament.

A second inhaler device 1′ according to the present invention is shown in FIGS. 17A-D of the accompanying drawings. Components of the second inhaler device 1′ are individually shown in FIGS. 18-30 of the accompanying drawings.

The second inhaler device 1′ is a modification of the first inhaler device 1 and has the same component parts as the first inhaler device 1 albeit slightly modified in some cases as shown in the accompanying drawings and as discussed in more detail below. It will be appreciated that the basic operation and interaction of the components in the second inhaler device 1′ is essentially the same as in the first inhaler device 1.

The principal differences between the first and second inhaler devices 1, 1′ are now discussed below.

Firstly, with regard to FIG. 18 of the accompanying drawings, it will be understood that the base 2′ of the second inhaler device 1′ differs from that of the first inhaler device 1 in that four apertures 100 are provided along an upper perimeter edge 102 of the base 2′ in order to receive four posts 104 extending downwardly from a lower perimeter edge 106 of the middle cover 4′. The apertures 100 are asymmetrically positioned along the perimeter edge 102 of the base 2′ and the posts 104 are arranged in corresponding positions along the perimeter edge 106 of the middle cover 4′ so that all four posts 104 can only be simultaneously inserted within the apertures 100 when the base 2′ and middle cover 4′ are correctly aligned relative to one another. Insertion of the posts 104 into the apertures 100 secures the base 2 and middle cover 4 together.

The dose counter wheel 26′ is further provided with an aperture 110 which is alignable with a further aperture 108 extending through the base 2′ shown in FIG. 21. It will be understood that, when the dose counter wheel 26′ is mounted on the first boss 28′, it may be rotated relative to the base 2′ and thereby positioned so that the apertures 108, 110 align. An element (not shown) may then be temporarily inserted through the aligned apertures 108, 110 so as to prevent relevant rotation between the dose counter wheel 26′ and the base 2′ during assembly and/or storage of the second inhaler device 1′.

The mouthpiece 8 of the second inhaler device 1′ also includes a mouthpiece opening 112 (through which medicament is inhaled) which is provided with three helical guide vanes 114 for imparting a swirl motion onto medicament/fluid to be inhaled. Other means for imparting a swirl motion on medicament/fluid may be provided.

Furthermore, the knob 20′ (which engages with the operating member 54′) is provided as a integral part of the mouth piece cover 10′ as shown in FIG. 20. In use of the second inhaler device 1′, the mouthpiece cover 10′ is detached from the housing of the inhaler device 1′ at one end thereof by disengaging a clip element 116 on the mouthpiece cover from a lip 118 provided by the base 2′ and middle cover 4′. In so doing, the mouthpiece cover 10′ is rotated from the housing. This rotation is permitted by means of a hinge arrangement provided by a flexible strip of material 12′ integrally connecting the mouthpiece cover 10′ and the knob 20′.

The operating member 54′ of the second inhaler device 1′ differs from the operating member 54 of the first inhaler device 1 in that the clip 120 is orientated so as to engage and thereby support the full depth of the knob 20′. In the first inhaler device 1, the knob 20 is merely supported at its centre and, accordingly, has a tendency to be pressed against the housing of the device at its upper and lower ends during use. The operating member 54′ of the second inhaler device also differs from that of the first inhaler device in that a second camming strip 130 is located below the aperture 68′. The second cam 130 is orientated so as to press the roller 72′ associated with the spiking component 22′ upwardly, thereby returning the support arm 90′ to an upper position within the housing (as the operating member 54′ is moved in a direction as indicated by arrow A in FIG. 25F). In this way, the arm 90′ is returned to its storage/retracted position by means of a camming action without the need for biasing means (such as the resilient elasticity of the arm 90′). In the second inhaler device 1′, the arm 90′ is returned to the retracted position by means of both the inherent elasticity of the arm 90′ and by the cam 130.

With reference to FIG. 29, the under side of the middle cover 4′ is shown wherein it can be seen that resilient clip elements 132 project downwardly from either side of aperture 94′ and are provided with bosses 134 for snap-fitting into apertures 136 of the arm 90′. During operation of the second inhaler device 1′, the arm 90′ rotates up and down about the bosses 134. When the arm 90′ is located in its upper-most position within the housing of the inhaler device 1′, the arm 90′ locates between two elements 138 projecting downwardly from the under-side of the middle cover 4′. In this way, the end of the arm 90′ distal to the apertures 136 is prevented from moving from side to side.

Furthermore, the middle cover 4′ is provided with four apertures 140 for receiving and retaining therein four pegs 142 projecting downwardly from the underside of the top cover 6′. In this way, the top cover 6′ is securable to the middle cover 4′.

Although the underside of the middle cover 4 of the first inhaler device 1 is not shown in the accompanying drawings, it comprises the same features (other than the posts 104) as those shown for the middle cover 4′ of the second inhaler device 1′.

Further minor differences between the first and second inhaler devices may be summarised as follows.

Firstly, the drive gear 30′ is provided with three apertures 150. Secondly, the centring mechanism 50′ of the second inhaler device 1′ has an associated roller 52′ provided with four apertures 154 extending thereto so as to provide radially extending spokes 156.

With reference to FIG. 27, a medicament cartridge 38′ is shown as comprising thirty doses of medicament. However, this cartridge 38′ is identical to the cartridge 38 of the first inhaler device 1. Also, the spiking component 22′ and associated roller 72′ of the second inhaler device 1′ are identical to the corresponding components 22, 72 of the first inhaler device 1.

With regard to materials of manufacture, the top cover 6′, middle cover 4′, drive gear 30′, dose counter 26′ and base 2′ may be manufactured from Acrylonitrile Butadiene Styrene (MF45). Also, the arm 90′, spike 22′ and associated roller 72′, operating member 54′, and centring mechanism 50′ and associated roller 52′ are manufactured from Acetyl co-polymer. Finally, the mouthpiece 8′ and associated cover 10′ are manufactured from polypropylene, and the medicament cartridge 38′ is manufactured from polystyrene (206). The corresponding components of the first inhaler device 1 may be manufactured from these same materials.

The present invention is not limited to the specific embodiment described above. Alternative arrangements and suitable materials will be apparent to a reader skilled in the art. For example, in an alternative embodiment, a dose counter is not provided. In such an inhaler, the counter wheel 26, teeth 34 and window 24 will not be required and may be omitted. It will be understood therefore that the dose counter is an optional feature. It will also be appreciated that the spiking component 22, 22′ can be made integrally with the arm 90, 90′ so that the spiking component 22, 22′ and arm 90, 90′ are a unitary item. The arm 90, 90′ may also be pivotally connected to the remainder of the inhaler (for example, by means of the connection of hooking element 90 with the middle cover 4). This pivotal connection may allow the arm 90, 90′ to move to and from the retracted/storage position without a bending or other deformation of the arm 90, 90′ itself. In such an arrangement, it will be understood that no spring bias is provided by virtue of the arm material bending/deforming. Alternative means for providing spring bias (for example, a separate leaf spring) will need to be provided if the arm is to be moved by means of a spring bias. Alternatively, the arm may be moved by means other than a spring bias arrangement (for example, a camming system) as explained above.

1. An inhaler device for dispensing multiple doses of medicament, the inhaler comprising means for receiving a medicament cartridge comprising a plurality of compartments containing medicament; medicament extraction facilitating means for locating adjacent or in a compartment of a received cartridge and thereby allowing an extraction of medicament from the compartment; and operating means moveable in first and second directions by a user; wherein the medicament extraction facilitating means is movable from a retracted position to an extraction position in response to movement of the operating means in the first direction, the extraction position of the medicament extraction facilitating means allowing extraction of medicament from said cartridge compartment upon inhalation by a user, and the retracted position allowing advancement of the medicament cartridge, and wherein the medicament cartridge is driven by the operating means so as to advance a cartridge compartment into a predetermined position relative to the medicament extraction facilitating means in response to movement of the operating means in the second direction.

2. An inhaler device according to claim 1, wherein the medicament extraction facilitating means is driven by the operating means so as to move the medicament extraction facilitating means from the retracted position to the extraction position.

3. An inhaler device according to claim 1, wherein the medicament extraction facilitating means moves from the extraction position to the retracted position in response to movement of the operating means in the second direction.

4. An inhaler device according to claim 3, wherein the medicament extraction facilitating means is driven by the operating means so as to move the medicament extraction facilitating means from the extraction position to the retracted position.

5. An inhaler device according to claim 2, wherein the medicament extraction facilitating means is driven through use of a cam arrangement.

6. An inhaler device according to claim 5, wherein a cam is provided on the operating means.

7. An inhaler device according to claim 6, wherein the cam is provided as a ramp surface which moves laterally in abutment with the medicament extraction facilitating means and thereby displaces the medicament extraction facilitating means.

8. An inhaler device according to claim 6, wherein the medicament extraction facilitating means is provided with a roller or bearing before abutment with the cam.

9. An inhaler device according to claim 1, further comprising biasing means to move the medicament extraction facilitating means from the extraction position to the retracted position.

10. An inhaler device according to claim 1, further comprising biasing means to move the medicament extraction facilitating means from the retracted position to the extraction position.

11. An inhaler device according to claim 9, wherein the biasing means comprises a resilient elastic member.

12. An inhaler device according to claim 11, wherein the resilient elastic member comprises an arm.

13. An inhaler device according to claim 1, wherein the medicament extraction facilitating means is provided on an arm.

14. An inhaler device according to claim 12, wherein one end of the arm is fixed to a housing of the inhaler device and the medicament extraction facilitating means is provided on a free end of the arm.

15. An inhaler device according to claim 14, wherein the arm and medicament extraction facilitating means are provided as a unitary component.

16. An inhaler device according to claim 12, wherein the arm is fixed to a housing of the inhaler device by means of a pivotal connection.

17. An inhaler device according to claim 1, wherein the medicament extraction facilitating means is provided with a tubular element for locating in a cartridge compartment when the medicament extraction facilitating means is located in the extraction position.

18. An inhaler device according to claim 17, wherein a free end of the tubular element is provided with a piercing portion.

19. An inhaler device according to claim 18, wherein the piercing portion is of a frusto-conical shape.

20. An inhaler device according to claim 17, wherein the tubular element is in fluid communication with a mouthpiece of the inhaler device when the medicament extraction facilitating means is located in the extraction position.

21. An inhaler device according to claim 17, wherein fluid communication between the tubular element and a mouthpiece of the inhaler device is prevented when the medicament extraction facilitating means is located in the retracted position.

22. An inhaler device according to claim 20, wherein the mouthpiece comprises means for imparting a swirl motion onto the medicament to be inhaled.

23. An inhaler device according to claim 22, wherein the means for imparting a swirl motion onto the medicament comprises three helical guide vanes.

24. An inhaler device according to claim 20, wherein the fluid communication is through an aperture or discontinuity provided in the operating means.

25. An inhaler device according to claim 24, wherein the aperture or discontinuity is aligned with a flow path through the mouthpiece when the medicament extraction facilitating means is in the extraction position.

26. An inhaler device according to claim 25, wherein the alignment of the aperture or discontinuity and the flow path through the mouthpiece is achieved by movement of the operating means in the first direction.

27. An inhaler device according to claim 24, wherein the aperture or discontinuity is not aligned with a flow path through the mouthpiece when the medicament extraction facilitating means is in the retracted position.

28. An inhaler device according to claim 27, wherein the aperture or discontinuity and the flow path through the mouthpiece are brought out of alignment by movement of the operating means in the second direction.

29. An inhaler device according to claim 1, wherein the medicament extraction facilitating means comprises a venturi.

30. An inhaler device according to claim 1, further comprising ratchet means for allowing the operating means to drive the medicament cartridge.

31. An inhaler device according to claim 30, wherein the operating means is provided with a ratchet tooth.

32. An inhaler device according to claim 30, wherein said ratchet tooth is arranged so as to engage in turn with each of a plurality of ratchet teeth provided on the medicament cartridge to advance the cartridge.

33. An inhaler device according to claim 1, wherein the cartridge compartment is advanced to a position adjacent the medicament extraction facilitating means.

34. An inhaler device according to claim 1, wherein said first direction of movement of the operating means is opposite to said second direction.

35. An inhaler device according to claim 1, wherein movement of the operating means in said first and second directions comprises a movement of the operating means along a part-circular path.

36. An inhaler device according to claim 35, wherein the part-circular path is concentrically positioned relative to an annular arrangement of medicament compartments of the medicament cartridge.

37. An inhaler device according to claim 1, wherein an element of the operating means extends through a slot in a housing of the inhaler device.

38. An inhaler device according to claim 37, wherein the slot is configured so as to allow movement of said element between opposite ends of the slot and thereby move the operating means in said first and second directions.

39. An inhaler device according to claim 37, wherein retaining means are provided for selectively preventing movement of said element of the operating means.

40. An inhaler device according to claim 39, wherein said element retaining means are provided on a cover for the mouthpiece.

41. An inhaler device according to claim 39, wherein said element retaining means comprise part of a cover for the mouthpiece.

42. An inhaler device according to claim 1, further comprising means for retaining a medicament cartridge in a required position.

43. An inhaler device according to claim 42, wherein the cartridge retaining means comprise a roller, wheel or bearing to press against the medicament cartridge whilst allowing movement of the cartridge.

44. An inhaler device according to claim 42, wherein the cartridge retaining means are sprung biased so as to press against the medicament cartridge.

45. An inhaler device according to claim 42, wherein the cartridge retaining means is made from delrin or acetal.

46. An inhaler device according to claim 1, further comprising means for driving a dose counter in response to movement of a medicament cartridge.

47. An inhaler device according to claim 46, wherein said drive means comprises one or more teeth or detents for engaging a medicament cartridge.

48. An inhaler device according to claim 47, wherein the drive means may further comprises a gear wheel for engaging means for displaying a dose count.

49. An inhaler device according to claim 1, wherein the medicament extraction facilitating means is formed from delrin or acetal.

50. An inhaler device according to claim 12, wherein the arm is a cantilever arm.
